(19) United States
(12) Patent Application Publication
(10) Pub. No.: US 2005/0107758 A1
(43) Pub. Date: May 19, 2005

(54) Ostomy comfort pad

(76) Inventors: Richard A. Hogan, Mt. Dora, FL (US); Ruth A. Hogan, Mt. Dora, FL (US)

Correspondence Address: Edward M. Livingston, 963 Trail Terrace Drive, Naples, FL, 34103, US

(21) Appl. No.: 10/712,557

(22) Filed: Nov. 13, 2003

(51) Int. Cl. A61M 1/00

(52) U.S. Cl. 604/327

(57) ABSTRACT

An ostomy comfort pad (1) that absorbs heat and moisture yet is still lightweight and not bulky. The pad (1) is sized slightly larger than an ostomy appliance (9) and has a similarly shaped body (4). A socket (6) is sized for the acceptance of an ostomy appliance collar (10) so as to surround the collar (10) when in use. The pad (1) has fasteners (2) located on the arms (3) of the pad (1) to secure the pad (1) to the ostomy belt (7). A clip-holder pocket (5) is located at the bottom of the pad (1) and is sized for the acceptance of the ostomy appliance clip (12). The pad (1) has a filling (8), preferably of pinewood fluff, so as to absorb the heat radiated from the excrements and an outer covering (13), preferably made of a polypropylene material, so as to be soft against the user's skin. The pad (1) is particularly well suited for ostomy appliance (9) users who dislike the irritation and discomfort caused by wearing ostomy appliances (9) and also dislike the added bulk that accompanies traditional ostomy appliance (9) covers.

### BACKGROUND OF THE INVENTION

This invention relates to ostomy appliances, also referred to as ostomy bags, and more particularly, an ostomy comfort pad which acts as an barrier to prevent irritation caused by the appliance to the user.

People who wear ostomy appliances often encounter many problems, both physically and mentally, regarding usage of the device. One of the most common problems associated with wearing an ostomy appliance is that because the appliance itself is generally made of thin plastic, heat transfers from the higher temperature excrement to the user's skin, thereby causing an uncomfortably warm sensation against the user's body. Additionally, the heat from the excrement causes the user's skin to sweat, thus causing moisture to come between the skin and the appliance. A further problem associated with wearing an ostomy appliance is when the appliance becomes full with liquid or excrement. When the user moves and the appliance is full, the appliance sways, causing the user to become embarrassed and concerned as there is a danger that the clip on the bottom can become loose and spill the contents of the appliance.

Although there are many types of covers for ostomy appliances which help to reduce the heat transfer, these appliances are unsanitary because they are not disposable. Additionally, these covers are often like drawstring appliances, producing bulges under the clothing so as to make the ostomy appliance visible to others. Furthermore, these covers are time consuming to remove and replace whenever a user needs to empty the appliance. As such, ostomy appliance users are left with the tough decision of choosing between the lesser of two embarrassments: either dealing with the uncomfortable feeling of warm bodily excrements against his or her skin or having a bulge located under the user's clothing, indicating to the public that the person is trying to hide something.

Thus, the present invention helps prevent the transfer of heat from excrements to the user's skin yet being lightweight, sanitary and not bulky.

The prior art includes the following United States patents:

| Patent No. | Inventor | Assignee | Filing Date | Issue Date |
|---|---|---|---|---|
| 5,653,701 | Millman | Desert Shield Trust | Nov. 2, 1995 | Aug. 5, 1997 |
| 5,626,570 | Gallo | N/A | Jun. 11, 1996 | May 6, 1997 |
| 5,026,362 | Willett | N/A | Jun. 1, 1990 | Jun. 25, 1991 |
| 2,205,270 | Perry | N/A | Mar. 20, 1939 | Jun. 18, 1940 |
| US2002/0010445A1 | Gunn | N/A | Jul. 19, 2001 | Jan. 24, 2002 |
| US2003/0105438A1 | Hostetler | N/A | Dec. 4, 2002 | Jun. 5, 2003 |
| 5.248,308 | von Emster | N/A | Jun. 29, 1992 | Sep. 28, 1993 |
| 5,607,412 | Brown | N/A | Jun. 6, 1995 | Mar. 4, 1997 |
| US6,186,989 B1 | Horie | Horiekikaku Co., Ltd. | Sep. 15, 1998 | Feb. 13, 2001 |

### SUMMARY OF THE INVENTION

The primary object of the present invention is to provide an ostomy comfort pad that eliminates the transfer of heat from the excrement in the appliance to an ostomy appliance user's skin.

A further object of the present invention is to provide an ostomy comfort pad that is lightweight.

An even further object of the present invention is to provide an ostomy comfort pad that is disposable.

A further object of the present invention is to provide an ostomy comfort pad that is not bulky.

An even further object of the present invention is to provide an ostomy comfort pad that attaches to an ostomy holding belt.

An even further object of the present invention is to provide an ostomy comfort pad that eliminates appliance shifting when the user moves.

An even further object of the present invention is to provide an ostomy comfort pad that acts as a moisture barrier.

An even further object of the present invention is to provide an ostomy comfort pad that can be used on ostomy appliances of varying lengths.

The present invention fulfills the above and other objects by providing an ostomy comfort pad that is worn between the ostomy appliance and a person's skin. The pad has an exterior surface preferably made of polypropylene with pine-fluff filling. A clip-holder pocket, sized slightly smaller than an ostomy appliance clip, is located at the bottom of the pad so as to hold the ostomy appliance clip, and thus appliance, in place when it is becoming full of liquids or when the user moves. Self-adhesive tabs or a hook and loop type fastener material are located at the top of the pad on the pad arms to secure the pad to a standard ostemy belt. The pad arms are of sufficient length to accommodate ostomy appliances of varying lengths.

The above and other objects, features, and advantages of the present invention should become even more readily apparent to those skilled in the art upon a reading of the following detailed description in conjunction with the drawings wherein there is shown and described illustrative embodiments of the invention.

### BRIEF DESCRIPTION OF THE DRAWINGS

This invention is described by appended claims in relation to a description of a preferred embodiment with reference to the following drawings which are explained briefly as follows:

FIG. 1 is a frontal view of the ostomy comfort pad of the present invention;

FIG. 2 is a side view of the embodiment of FIG. 1;

FIG. 3 is a frontal cutting view of the embodiment of FIG. 1 secured to a standard ostomy belt;

FIG. 4 is frontal view of the embodiment of FIG. 1 in use; and

FIG. 5. is a downward cross-sectional view along the lines 5-5 of the embodiment of FIG. 1.

### DESCRIPTION OF THE PREFERRED EMBODIMENT

Listed numerically below with reference to the drawings are terms used to describe features of this invention. These terms and numbers assigned to them designate the same features throughout this description.

1. ostomy comfort pad

2. fastener

3. arm

4. body section

5. clip-holder pocket

6. socket

7. ostomy belt

8. filling

9. ostomy appliance

10. ostomy appliance collar

11. ostomy appliance tab

12. ostomy appliance clip

13. outer cover

14. top section

15. bottom section

16. front section

17. back section

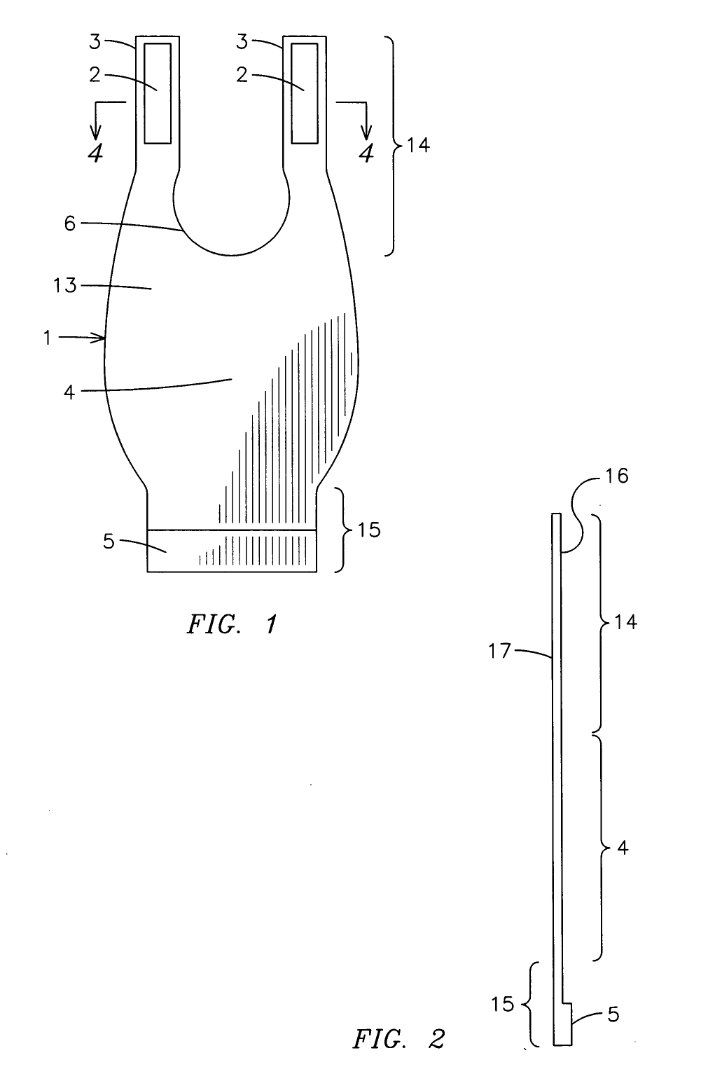

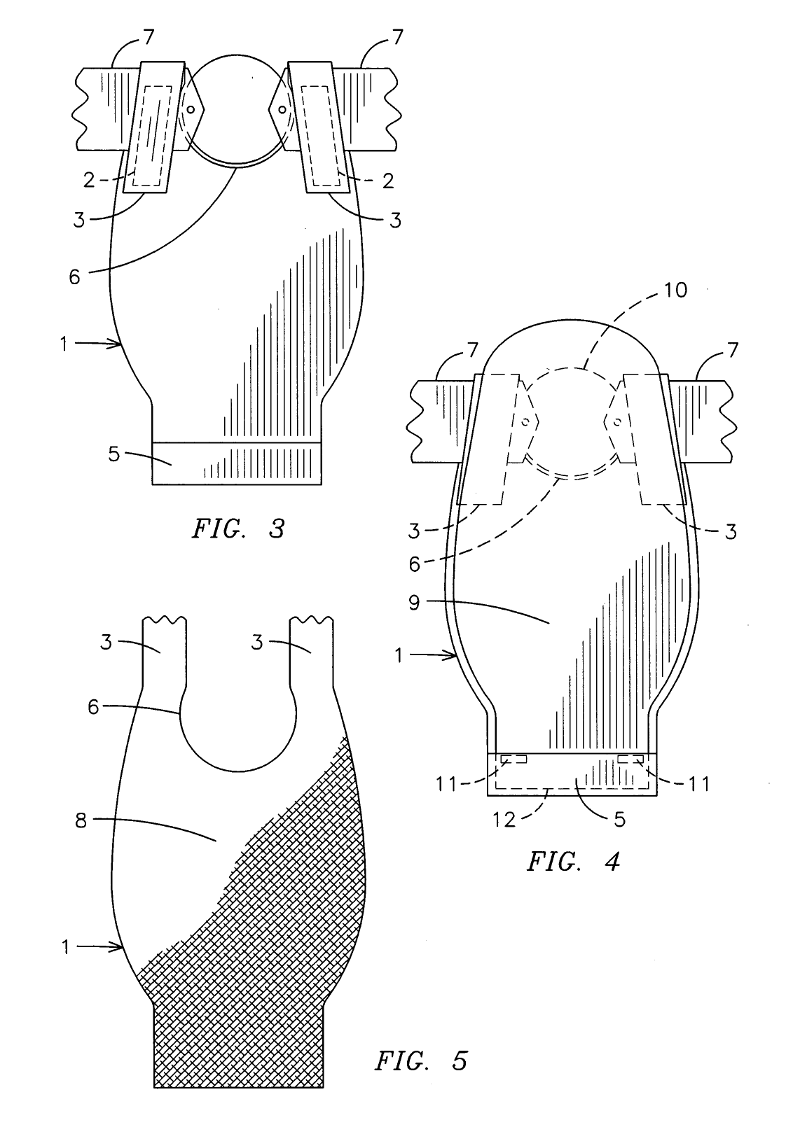

With reference to FIGS. 1 through 4, views of the present invention are shown. The ostomy comfort pad 1 has front section 16 and a back section 17, each with a top section 14, a body section 4 and a bottom section 15. The body section 4 and the bottom section 15 are sized and shaped slightly larger than an ostomy appliance 9 while the top section 14 has two arms 3. A socket 6 is sized for the acceptance of an ostomy appliance collar 10 so as to surround the lower portion of the collar 10 when in use. The pad 1 has fasteners 2, preferably made of self-adhesive tabs or a hook-and-loop type material, located on the arms 3 of the front section of the pad 16 to secure the pad 1 to an ostomy belt 7. A clip-holder pocket 5 is located on the front section 16 of the pad at the bottom section 15 and is sized for the acceptance of an ostomy appliance clip 12 and ostomy appliance tab 11.

With reference to FIG. 5, a downward cross-sectional view along lines 5-5 of the embodiment of FIG. 1 is shown. A thin layer of filling 8, preferably made of pinewood fluff, located within the pad 1 absorbs the heat given off from excrements.

For an ostomy appliance user to use the ostomy comfort pad 1, the user first lifts the ostomy appliance 9 so as to expose the ostomy appliance collar 10. The user then takes the arms 3 of the pad 1, fastener 2 sides out, and places the arms 3 under the ostomy belt 7. The user then takes the pad 1, and lifts it so as to place the socket 6 of the pad 1 flush against the lower portion of the ostomy appliance collar 10. The user then secures the fasteners 2 to the belt 7 by either removing the self-adhesive covering and pressing down on the fastener 2 or by pressing down on the hook-and-loop type fastener. Finally, the user places the ostomy appliance clip 12 into the clip-holder pocket 5.

When the appliance 9 needs to be emptied, the user lifts the ostomy appliance clip 12 out of the pocket 5, lifts the ostomy clip tabs 11 and drains the appliance 9. The user now attaches a new ostomy comfort pad 1 to the ostomy belt 7 to promote sanitary measures.

The use of the present invention will prevent ostomy appliance users from experiencing the uncomfortable feeling of having warm excrements against their skin without adding noticeable bulk under the user's clothing. Additionally, the use of the present invention will promote more sanitary methods regarding ostomy appliance use.

It is to be understood that while a certain form of the invention is illustrated, it is not to be limited to the specific form or arrangement of parts herein described and shown. It will be apparent to those skilled in the art that various changes may be made without departing from the scope of the invention and the invention is not be considered limited to what is shown and described in the specification and drawings.

1. An ostomy comfort pad comprising:
a front section and a back section having edges;
said front section is attached to said back section along said edges;
said front and back sections having a top section, a body section and a bottom section;
said top sections having at least one arm;
said at least one arm having a fastener;
said body sections having a predetermined length;
said body sections having a cut-out;
said front bottom section having a pocket;
said front and back sections having a cover; and
a filling located between said front and back sections.

2. The ostomy comfort pad of claim 1 wherein:
said top sections have two arms; and
said front top, body and bottom sections are the same size and shape as said back top, body and bottom sections.

3. The ostomy comfort pad of claim 2 wherein:
said fastener is a hook and loop type material; and
said hook and loop type material is located on said front section.

4. The ostomy comfort pad of claim 2 wherein:
said fastener is an adhesive tab; and
said adhesive tab is located on said front section.

5. The ostomy comfort pad of claim 2 wherein:
said arms have a predetermined length; and
said arm lengths is at least half the length of the body sections.

6. The ostomy comfort pad of claim 2 wherein:
said cut-out is sized and shaped for the acceptance of an ostomy appliance collar.

7. The ostomy comfort pad of claim 2 wherein:
said pocket is sized and shaped for the acceptance of an ostomy appliance clip.

8. The ostomy comfort pad of claim 1 wherein:
said cover is polypropylene.

9. The ostomy comfort pad of claim 1 wherein:
said filling is pinewood fluff.

10. The ostomy comfort pad of claim 1 wherein:
said body sections are sized slightly larger than an ostomy appliance; and
said bottom sections are sized slightly larger than an ostomy appliance.
